(19) United States
(12) Patent Application Publication
(10) Pub. No.: US 2019/0082973 A1
(43) Pub. Date: Mar. 21, 2019

(54) HEALTH STATUS MONITOR

(71) Applicant: FirstEnergy Ventures Corp., Akron, OH (US)

(72) Inventor: Aimee Michelle Knisely, Mogadore, OH (US)

(73) Assignee: FirstEnergy Ventures Corp., Akron, OH (US)

(21) Appl. No.: 16/137,006

(22) Filed: Sep. 20, 2018

Related U.S. Application Data
(60) Provisional application No. 62/560,729, filed on Sep. 20, 2017.

(51) Int. Cl. A61B 5/0205; A61B 5/00; G16H 50/30

(52) U.S. Cl. CPC A61B 5/0205; A61B 5/0022; A61B 5/6803; A61B 5/1118; G16H 50/30; A61B 5/7282; A61B 5/681

(57) ABSTRACT

One or more techniques and/or systems are disclosed for monitoring a health status of an individual. A person responsible for an individual's well-being may use such a system or technique to monitor them for possible health risks, such as heat stroke. A data reading device can be disposed in a non-invasive wearable, such as a wristband or headband, to be worn by the monitored individual. The data reading device can monitor data indicative of the target individual's health status, such as temperature, pulse rate and sweat conductivity. The health status data can be compared to pre-determined baseline thresholds for the individual, and a notification can be provided if the thresholds are exceeded.

### CROSS-REFERENCE TO RELATED APPLICATIONS

This application claims priority to U.S. Provisional Patent Application Ser. No. 62/560,729, entitled HEALTH STATUS MONITOR, filed Sep. 20, 2017, which is incorporated herein by reference.

### BACKGROUND

Athletes, construction workers, and others that may work, play, or otherwise are exposed to elevated heat and humidity may be at risk for heat related injury. For example, heat stress and heat stroke are conditions that may lead to reduced performance, injury, medical emergency, and even death. Identifying health related symptoms early may help mitigate some of these potential issues.

### SUMMARY

This Summary is provided to introduce a selection of concepts in a simplified form that are further described below in the Detailed Description. This Summary is not intended to identify key factors or essential features of the claimed subject matter, nor is it intended to be used to limit the scope of the claimed subject matter.

One or more techniques and systems described herein can be utilized to enable a person responsible for an individual's well-being to monitor them for possible health risk of heat stroke. For example, a manager/supervisor, coach, trainer, or other may be responsible for the well-being of a participant, athlete, employee, client, etc. In one implementation, a data reading device can be disposed in a non-invasive wearable, such as a wristband or headband, which can be worn by the monitored individual on the wrist or forehead. The data reading device can monitor data indicative of an individual's health status, such as temperature, pulse rate and sweat conductivity. In this implementation, the data reading device can receive (e.g., wirelessly) the data and make it available to computing device, such as a tablet, smartphone, other portable, or computer. The data provided by the data reader can be accessed by an application (e.g., app) resident of the computing device. Predetermined threshold information, such as standard body temperature, typical heart rate and sweat conductivity information for each individual, can be pre-loaded into the app. In one implementation, if the data indicative of the individual's health status surpasses the threshold information for the individual, the app may identify an alert status (e.g., an alarm), and provide a notification with information, such as to the receiving device, and/or the responsible person. As an example, the responsible person may be able to take corrective action to reverse the threat of heat stroke.

In one implementation of a system for monitoring a health status of an individual, one or more health status sensors can be disposed in a wearable component to be worn by a target individual. Further, in one implementation, such a system can comprise a health status data reader communicatively coupled with the one or more sensors to receive data indicative of a health status of the target individual from the one or more sensors. The example system can additionally comprise a health status computing component wirelessly, communicatively coupled with the data reader to receive data indicative of the health status of the target individual from the data reader to identify if the health status of the individual exceeds a predetermined health status threshold. The system for monitoring a health status of an individual can also comprise a notification component communicatively coupled with the computing component to provide a notification indicative of the health status of the target individual exceeding the predetermined health status threshold.

To the accomplishment of the foregoing and related ends, the following description and annexed drawings set forth certain illustrative aspects and implementations. These are indicative of but a few of the various ways in which one or more aspects may be employed. Other aspects, advantages and novel features of the disclosure will become apparent from the following detailed description when considered in conjunction with the annexed drawings.

### BRIEF DESCRIPTION OF THE DRAWINGS

FIG. 1 is a perspective view illustrating an example implementation of one or more portions of the systems described herein.

FIG. 2 is a perspective view illustrating an example implementation of one or more portions of the systems described herein.

FIG. 3 is a perspective view illustrating an example implementation of one or more portions of the systems described herein.

FIG. 4 is a perspective view illustrating an example implementation of one or more portions of the systems described herein.

FIG. 5 is an illustration of a perspective view of an example implementation of one or more portions of one or more systems described herein.

FIG. 6 is an illustration of a perspective view of an example implementation of one or more portions of one or more systems described herein.

FIG. 7 is an illustration of a perspective view of an example implementation of one or more portions of one or more systems described herein.

FIG. 8 is an illustration of a perspective view of an example environment where on or more implementations of one or more portions of one or more systems described herein.

FIG. 9 is an illustration of a perspective view of an example implementation of one or more portions of one or more systems described herein.

FIGS. 10A-10D are illustrations of an example implementation of one or more portions of one or more systems described herein.

FIG. 11 is a schematic diagram illustrating one implementation of a system for monitoring a health status of an individual.

### DETAILED DESCRIPTION

The claimed subject matter is now described with reference to the drawings, wherein like reference numerals are generally used to refer to like elements throughout. In the following description, for purposes of explanation, numerous specific details are set forth in order to provide a thorough understanding of the claimed subject matter. It may be evident, however, that the claimed subject matter may be practiced without these specific details. In other instances, structures and devices are shown in block diagram form in order to facilitate describing the claimed subject matter.

A system may be devised that can monitor an individual's health status, and provide notifications to the individual, and/or a person responsible for that individual, in the event that the health status data indicates an alert condition. In one implementation, a wearable device can comprise one or more sensors that detect a wearer's temperature, heart rate, and/or sweat conductivity. Such as device can be comprised of a light weight and flexible wearable, for example, which may be comfortably worn by athletes, employees, participants in events, etc. As an example, the wearable can comprise a head band, an arm band, a chest band, a patch applied to the skin, a shirt, or a hat.

In one implementation, the wearable may comprise a data device, comprising a health status data reader, which receives data indicative of the wearer's body temperature, heart rate and sweat conductivity, such as provided by the one or more sensors. Further, such as system can comprise an application (e.g., remotely from the wearable) disposed on a computing device (e.g., portable) that receives and processes the data indicative of the wearer's health status. As an example, the app can provide for the user to enter a base reading of the wearer's health status (e.g., vitals). In one example, the wearable device can be worn by the individual wearer prior to activity to identify base readings, and/or determine that the base readings are appropriate for the individual.

In one implementation, the application (app) can be pre-loaded on a portable (e.g., handheld) data receiving device, such as a smartphone, tablet, portable computing device, or computer. In this implementation, the wearable device can monitor the individual wearer (e.g., continuously or periodically reading sensor data from the sensors) while the wearer is performing the expected activity (e.g., sports, work, other activities). In this implementation, for example, pre-determined thresholds (e.g., health balanced presets limits) can be identified for the individual wearer, such as by the app, a health professional, and/or other available data. As an example, when data indicative of the wearer's health status, received by the data device, exceeds a preset threshold, the app loaded on the receiving device can provide a notification (e.g., alert) that a threshold has been exceeded. In one implantation, an alert (e.g., visual and/or auditory) can be provided on the receiving device (e.g., to the wearer) and/or a notification with details can be displayed on the portable receiving device. In this example, the user of the receiving handheld device, such as the responsible person, may be alerted to take time relevant action to mitigate the symptoms that lead to heat related illness, for example.

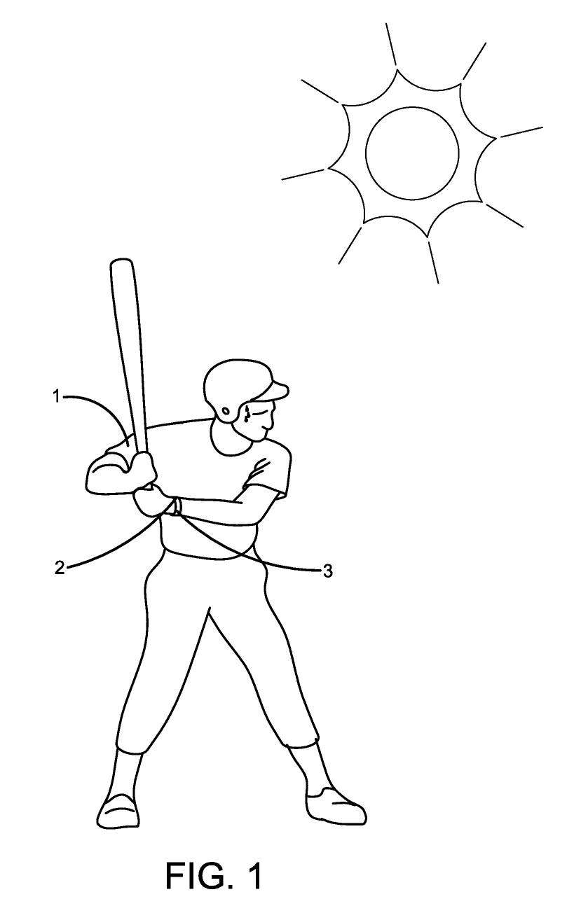

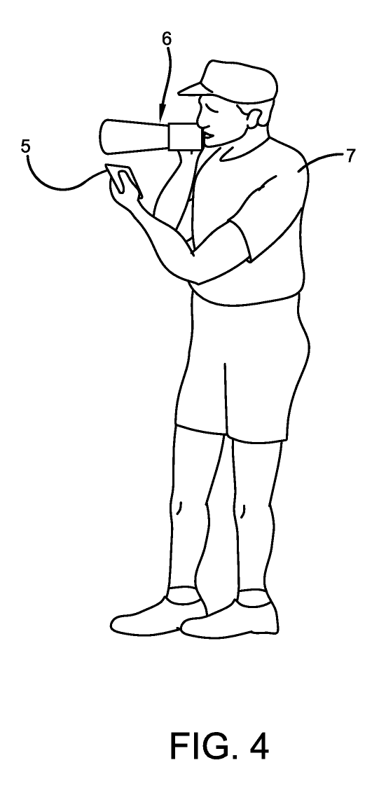

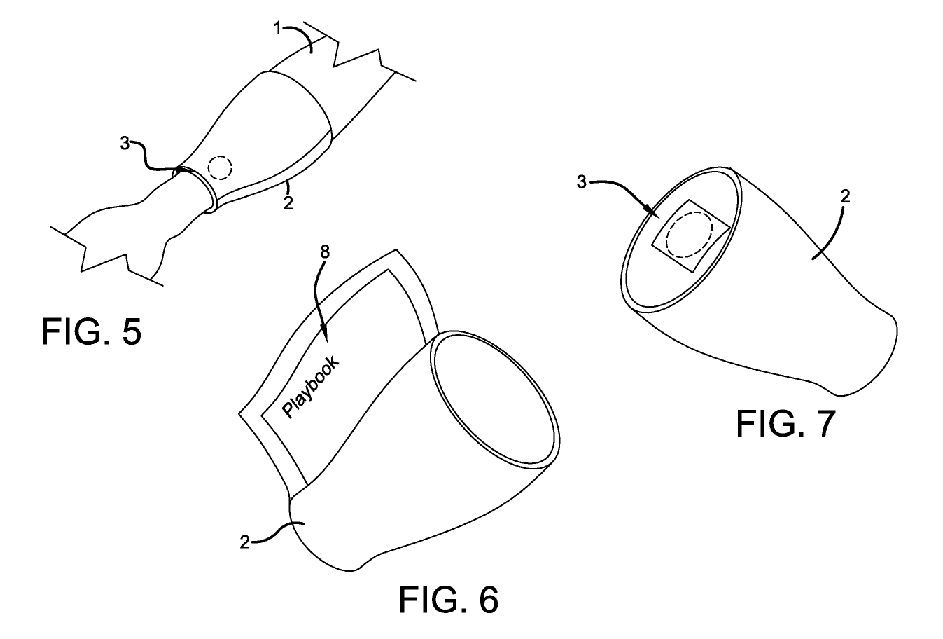

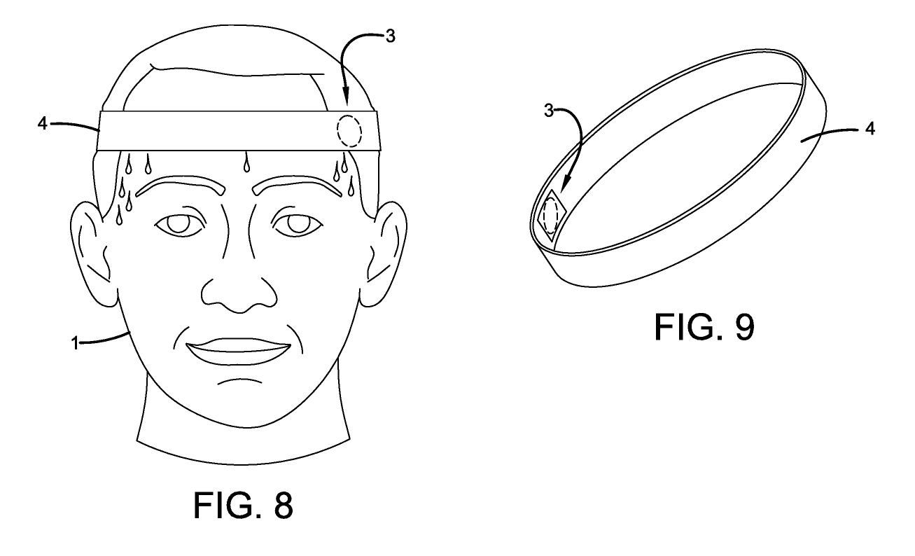

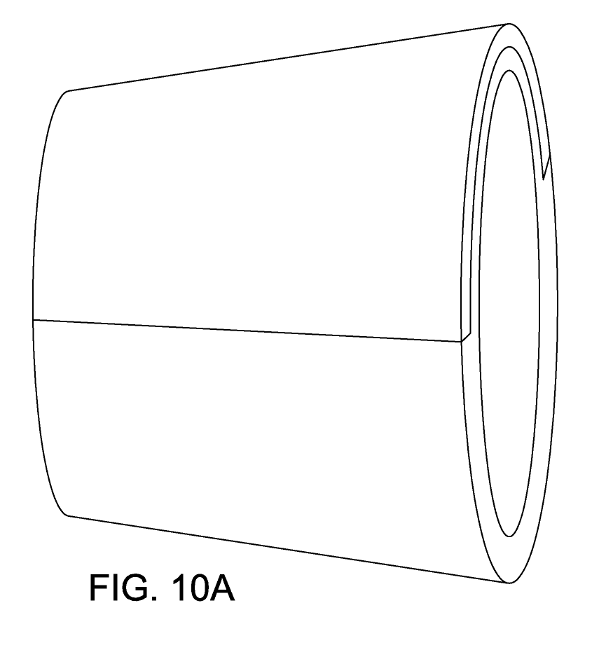

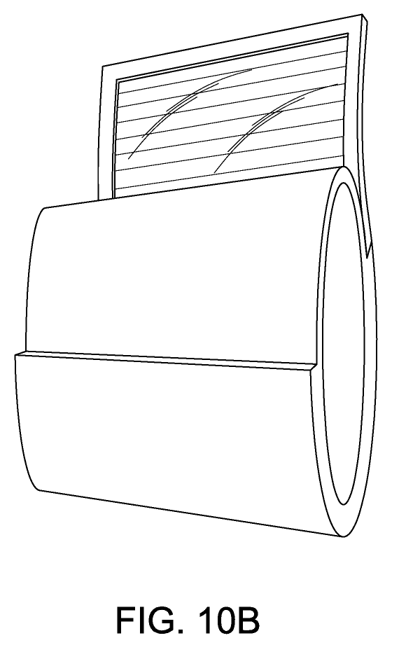

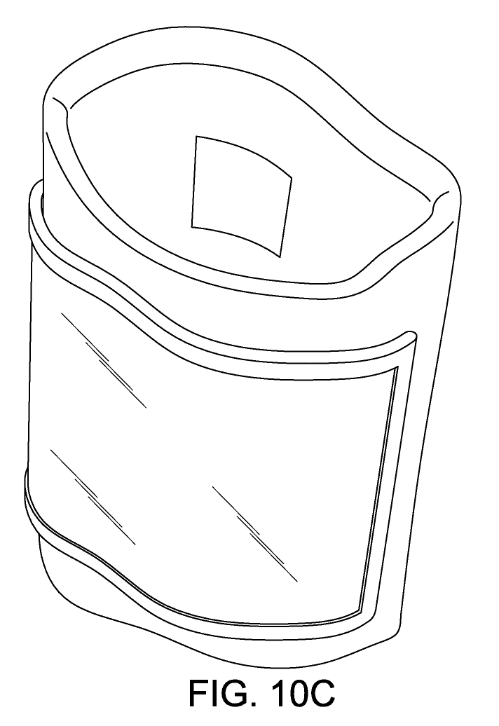

As an illustrative example, with reference to the FIGS. 1-9, an athlete/employee/monitored individual 1 can wear a non-invasive wearable wristband 2, which may or may not include a playbook 8, or a non-invasive wearable headband 4, during exercise/activities in which they are exposed to a high temperature environment. In this example, the non-invasive wearable wristband 2, and/or the non-invasive headband 4 can comprise a data reading device 3 (e.g., on-board), which can utilize one or more sensors to monitor the health status of the wearer, such as body temperature, pulse rate and sweat conductivity, and/or blood oxygen level. In one implementation, data indicative of the health status of the wearer can be received by a removable, cordless, chargeable data reading device 3.

Further, preset, health status thresholds can be determined for the individual wearer, such as an athlete/employee 1. In one implementation, data indicative of baseline health statistics (e.g., baseline vitals) can be loaded onto the computing device, to be used by the resident application 6. For example, the app can be installed onto a handheld receiving device 5 (e.g., computing device) such as a tablet, smartphone or computer.

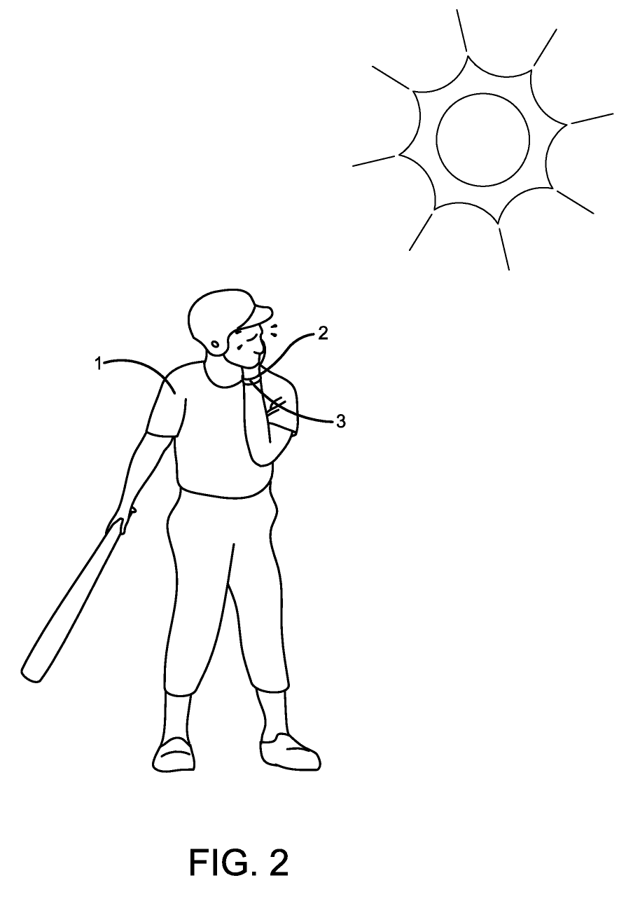

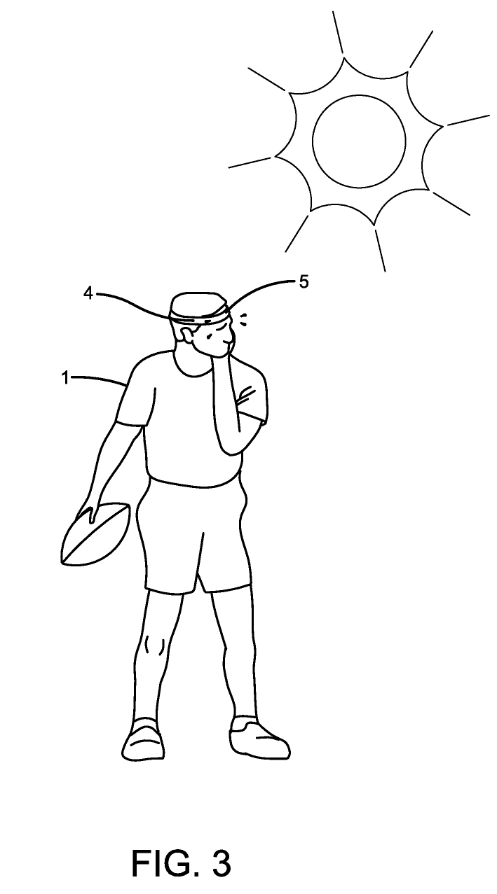

The wearable device 2, 4 can be placed on the individual wearer 1, and powered on. The wearable device 2, 4 can begin monitoring the health status of the wearer, such as the body temperature, heart rate and sweat conductivity. In this illustrative example, in FIGS. 2 and 3, the individual wearer 1 may begin to physically perform a task, such as one taking place in a high temperature environment or confined area, or wearing heat retaining clothing. As an example, increased physical activity may result in changes in vital health status indictors, such as body temperature, heart rate and sweat conductivity. In this example, the data reading device 3 may be able to identify these changes, and the preset thresholds can be utilized to determine if the individual wearer may have reached a threshold that is indicative of an alert condition, such as elevated body temperature (e.g., over one-hundred and four degrees Fahrenheit), erratic or increased heartbeat, increased or no sweat conductivity, and/or low blood-oxygen levels. As one example, these conditions may comprise alert conditions, which may be indicative of an impending heat-related illness, such as heat stress or heat stroke conditions.

In this illustrative example, the responsible person 7 (e.g., coach/manager/supervisor) may be the user in control of the portable computing device 5 (e.g., handheld device). In one example, when a baseline health statistic threshold for the individual wearer has been exceeded, the data reading device 3 can provide the sensor readings to the handheld device 5 (e.g., or to an on-board health status computing component, which can send a notification). As one example, the notification can result in an alert, such as an audible and/or visual alert on the portable device. Further, the notification can result in a visual indicator to the responsible person 7 of the identified health status of the wearer, on the handheld device 5.

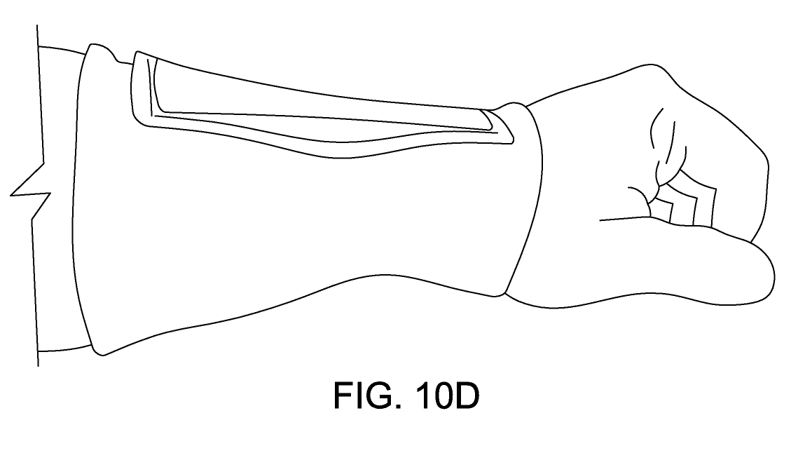

FIGS. 10A, 10B, 10C, and 10D illustrate an example implementation of a wearable component that may be utilized to implement one or more portions of one or more systems described herein. For example, a wearable portion of the systems described herein may comprise an arm band, such as a wrist or forearm band (e.g., as illustrated). In one example, the wrist or forearm band can comprise one or more sensors that may be utilized to detect one or more health status indicators of the wearer. As an example, the one or more sensors may be disposed in an interior portion of the wearable (e.g., or any appropriate location), with a health status reader portion exposed in the interior for contact or proximity with the wearer's skin. Further, in one implementation, the wearable device can comprise a data communication device, such as a wireless communications system (e.g., Bluetooth, Wi-Fi, or other wireless communications systems). Additionally, in one implementation, the wearable can comprise another component for use by the wearer, such as a note book portion (e.g., used for reading plays by a football player).

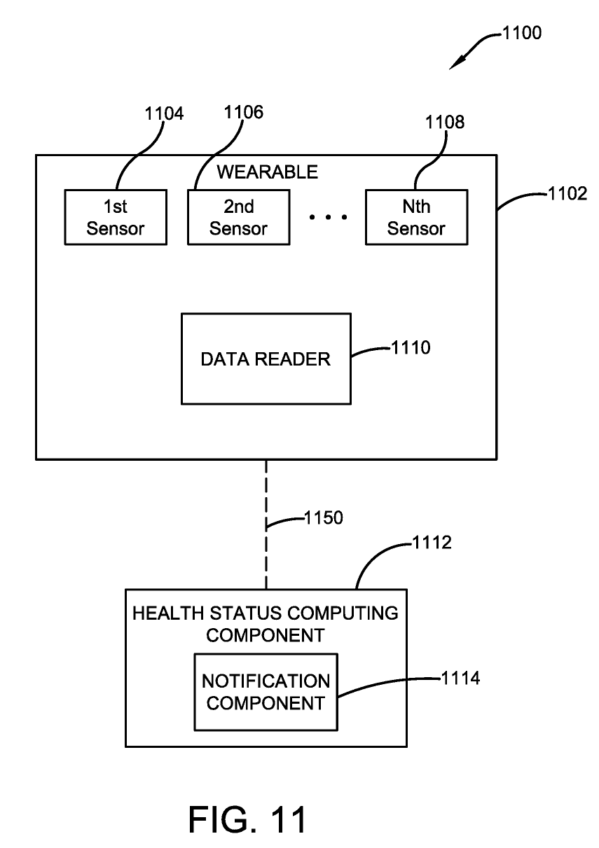

FIG. 11 is a schematic diagram illustrating one implementation of a system 1100 for monitoring a health status of an individual. In this example, the system 100 can comprise a wearable component 1102. As an example the wearable component can be configured to be worn a target individual, such as a person that may be subjected to conditions that may lead to a heat related illness or injury. Such wearables can comprise a headband, arm band, chest band, a patch applied to the skin, a shirt, or a hat. In this example, the wearable should be able to be worn next to the skin of the target individual.

Further, the example system 100 can comprise a plurality of sensors 1104, 1106, 1108 disposed in/on the wearable component. For example, as described herein, the plurality of sensors can comprise a body temperature sensor, a heart rate sensor, a blood oxygen sensor, and/or a perspiration conductivity sensor. The body temperature sensor can monitor the target individual's body temperature; the heart rate sensor can monitor the target individual's heart rate; the blood oxygen sensor can monitor the target individual's blood-oxygen level; and the perspiration conductivity sensor can monitor the conductivity of the target individual's perspiration at the skin's surface.

Additionally, the example system 1100 can comprise a health status data reader 1110. In one implementation, the health status data reader 1110 can be disposed in/on the wearable component, and communicatively coupled with the plurality of sensors 1104, 1106, 1108. The health status data reader 1110 can be used to receive data indicative of a health status of the target individual from one or more of the plurality of sensors 1104, 1106, 1108. The data indicative of a health status can comprise data regarding the temperature, heart rate, blood-oxygen level, and/or conductivity, for example.

In this system 1100, a health status computing component 1112 can be communicatively coupled (e.g., wirelessly or wired) with the data reader 1110 to receive the data indicative of the health status of the target individual from the data reader 1110. In one implementation, the health status computing component 1112 can comprise a remote computing device, such as a mobile device, to receive the data. In another implementation, the health status computing component 1112 can comprise a local device, such as disposed on the target individual, or on the wearable. The health status computing component 1112 can be used to determine if the data indicative of the health status of the target individual exceeds a predetermined health status threshold.

In one implementation, the system 100 can comprise a notification component 1114 that is communicatively coupled with the health status computing component 1112. In one implementation, the notification component 1114 can comprise an application or portion thereof that is resident on the health status computing component 1112. The notification component 1114 can provide a notification indicative of the health status of the target individual exceeding the predetermined health status threshold. For example, the notification component 1114 may notify the health status computing component 1112, or the notification may be transmitted to a separate remote device, such as to a user who is responsible for the target individual's health status (e.g., trainer, coach, manager, supervisor, etc.). In one example, an alert can be generated to alert the person responsible for the target individual, to alert them to the condition indicated for the target individual.

Moreover, the word “exemplary” may be used herein to mean serving as an example, instance or illustration. Any aspect or design described herein as “exemplary” is not necessarily to be construed as advantageous over other aspects or designs. Rather, use of the word exemplary is intended to present concepts in a concrete fashion. As used in this application, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Further, At least one of A and Band/or the like generally means A or B or both A and B. In addition, the articles “a” and “an” as used in this application and the appended claims may generally be construed to mean “one or more” unless specified otherwise or clear from context to be directed to a singular form.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

As used in this application, the terms “component,” “module,” “system,” “interface,” and the like are generally intended to refer to a computer-related entity, either hardware, a combination of hardware and software, software, or software in execution. For example, a component may be, but is not limited to being, a process running on a processor, a processor, an object, an executable, a thread of execution, a program and/or a computer. By way of illustration, both an application running on a controller and the controller can be a component. One or more components may reside within a process and/or thread of execution and a component may be localized on one computer and/or distributed between two or more computers.

Furthermore, the claimed subject matter may be implemented as a method, apparatus or article of manufacture using standard programming and/or engineering techniques to produce software, firmware, hardware or any combination thereof to control a computer to implement the disclosed subject matter. The term “article of manufacture” as used herein is intended to encompass a computer program accessible from any computer-readable device, carrier or media. Of course, those skilled in the art will recognize many modifications may be made to this configuration without departing from the scope or spirit of the claimed subject matter.

Also, although the disclosure has been shown and described with respect to one or more implementations, equivalent alterations and modifications will occur to others skilled in the art based upon a reading and understanding of this specification and the annexed drawings. The disclosure includes all such modifications and alterations and is limited only by the scope of the following claims. In particular regard to the various functions performed by the above described components (e.g., elements, resources, etc.), the terms used to describe such components are intended to correspond, unless otherwise indicated, to any component which performs the specified function of the described component (e.g., that is functionally equivalent), even though not structurally equivalent to the disclosed structure which performs the function in the herein illustrated exemplary implementations of the disclosure. In addition, while a particular feature of the disclosure may have been disclosed with respect to only one of several implementations, such feature may be combined with one or more other features of the other implementations as may be desired and advantageous for any given or particular application. Furthermore, to the extent that the terms “includes,” “having,” “has,” “with,” or variants thereof are used in either the detailed description or the claims, such terms are intended to be inclusive in a manner similar to the term “comprising.”

The implementations have been described, hereinabove. It will be apparent to those skilled in the art that the above methods and apparatuses may incorporate changes and modifications without departing from the general scope of this invention. It is intended to include all such modifications and alterations in so far as they come within the scope of the appended claims or the equivalents thereof.

What is claimed is:

1. A system for monitoring a health status of an individual, comprising:
one or more health status sensors disposed in a wearable component configured to be worn by a target individual;
a health status data reader communicatively coupled with the one or more sensors to receive data indicative of a health status of the target individual from the one or more sensors;
a health status computing component communicatively coupled with the data reader to receive the data indicative of the health status of the target individual from the data reader, and to determine if the data indicative of the health status of the target individual exceeds a predetermined health status threshold; and
a notification component communicatively coupled with the health status computing component to provide a notification indicative of the health status of the target individual exceeding the predetermined health status threshold.

2. The system of claim 1, the one or more health status sensors comprising one or more of:
a body temperature sensor;
a heart rate sensor;
a blood oxygen sensor; and
a perspiration conductivity sensor.

3. The system of claim 1, comprising a health status monitor disposed on a computing device wirelessly communicatively coupled with the health status computing component to receive the notification indicative of the health status of the target individual.

4. The system of claim 1, comprising a health status monitor disposed on the health status computing component to provide an alert upon receiving the notification indicative of the health status of the target individual.

5. The system of claim 1, the wearable component comprising one of:
a head band;
an arm band;
a chest band;
a patch;
a shirt; and
a hat.

6. The system of claim 1, the health status data reader comprising one of:
a wireless communication component to wirelessly transmit the data indicative of a health status of the target individual to the health status computing component; and
a wired communication component to couple with the health status computing component using a hard line to transmit the data indicative of a health status of the target individual.

7. The system of claim 1, the health status data reader comprising a removable component comprising a rechargeable power source.

8. The system of claim 1, the predetermined health status threshold comprising one or more of:
a threshold body temperature exceeding a predetermined temperature zone outside a baseline temperature for the target individual;
a threshold heartrate exceeding a predetermined heartrate zone outside a baseline heartrate for the target individual;
a threshold conductivity level exceeding a predetermined conductivity zone outside a baseline conductivity for the target individual; and
a threshold blood oxygen level exceeding a predetermined blood oxygen level zone outside a baseline conductivity for the target individual.

9. A method for monitoring a health status of an individual, comprising:
placing a wearable device on a target individual, the wearable device comprising one or more health status sensors, and a health status data reader communicatively coupled with the one or more sensors to receive data indicative of a health status of the target individual from the one or more sensors;
monitoring the health status of the target individual using the one or more sensors, and reading data indicative of the health status of the target individual from the one or more sensors;
transmitting the data indicative of the health status of the target individual to a health status computing component to identify if the health status of the individual exceeds a predetermined health status threshold; and
providing a notification indicative of the health status of the target individual exceeding the predetermined health status threshold.

10. The method of claim 9, the monitoring the health status of the target individual using the one or more sensors comprising monitoring one or more health status indictors comprising:
a body temperature of the target individual;
a heart rate of the target individual;
a blood oxygen level of the target individual; and
a perspiration conductivity level of the target individual.

11. The method of claim 10, identifying if the health status of the individual exceeds a predetermined health status threshold comprising:
determining if one or more of the health status indictors exceed its respective threshold; or
determining if a combination of one or more of the health status indictors exceeds a combination threshold.

12. The method of claim 10, comprising determining a baseline for the target individual for the respective one or more health status indictors.

13. The method of claim 9, providing the notification comprising providing the notification to a health status monitor disposed on a computing device wirelessly communicatively coupled with the health status computing component to receive the notification indicative of the health status of the target individual.

14. The method of claim 9, comprising providing an alert by a health status monitor disposed on the health status computing component upon receiving the notification indicative of the health status of the target individual.

15. The method of claim 9, placing the wearable device on the target individual comprising placing one or more of the following on the target individual:
a head band;
an arm band;
a chest band;
a patch;
a shirt; and
a hat.

16. The method of claim 9, comprising one of:
using the health status data reader to wirelessly transmitting the data indicative of a health status of the target individual to the health status computing component; and
using the health status data reader to couple with the health status computing component using a hard line to transmit the data indicative of a health status of the target individual.

17. The method of claim 9, comprising recharging a power source disposed in the health status reader.

18. A wearable device for monitoring a health status of an individual, comprising:
one or more health status sensors respectively to monitor a health status indicator of a target individual, the one or more health status sensors selected from:
a temperature monitor to measure body temperature;
a heart rate monitor to measure heart rate;
a blood oxygen monitor to measure blood oxygen; and
a perspiration conductivity monitor to measure perspiration conductivity;
a health status data reader communicatively coupled with the one or more sensors to receive data indicative of one or more health status indicators of the target individual from the one or more sensors, the health status data reader comprising a rechargeable power source;
a data transmission component to transmit the data indicative of one or more health status indicators to a remote health status computing component, the health status computing component to identify if the health status of the individual exceeds a predetermined health status threshold, and to provide a notification if one or more of the health status indicators of the target individual exceed a predetermined health status threshold.

19. The device of claim 17, comprising one of:
a head band to wear on the target individual's head;
an arm band to wear on one of the target individual's arms;
a chest band to wear on the target individual's chest;
a patch to apply to the target individual's skin;
a shirt to wear on the target individual's torso; and
a hat to wear on the target individual's head.

20. The device of claim 17, the health status data reader comprising one of:
a wireless communication component to wirelessly transmit the data indicative of a health status of the target individual to the health status computing component; and
a wired communication component to couple with the health status computing component using a hard line to transmit the data indicative of a health status of the target individual.
